(19) United States
(12) Patent Application Publication
(10) Pub. No.: US 2026/0124075 A1
(43) Pub. Date: May 7, 2026

(54) INJURY PREVENTING EAR PRESSURE RELIEF MASK

(71) Applicant: Zachary Kimura, San Jose, CA (US)

(72) Inventor: Zachary Kimura, San Jose, CA (US)

(21) Appl. No.: 19/177,745

(22) Filed: Apr. 14, 2025

Related U.S. Application Data
(60) Provisional application No. 63/633,097, filed on Apr. 12, 2024.

(51) Int. Cl. A61F 11/00

(52) U.S. Cl. CPC A61F 11/00

(57) ABSTRACT

An overpressure mitigation mask intended to relieve ear pressure has a connector comprising a barb extrusion mated to an elastomeric low durometer tube. The low durometer tube expands when exposed to higher pressures, which prevents harm to the wearer of the mask who otherwise would absorb the pressure. The mask includes a magnetic connection between the connector piece and the mask which will disconnect when exposed to higher pressures, without which the break point located at the magnetic connector may potentially harm the user.

### CROSS REFERENCE TO RELATED APPLICATIONS

This application claims the benefit of U.S. Provisional Application No. 63/633,097 filed on Apr. 12, 2024.

### BACKGROUND OF THE INVENTION

Many prior art masks and aspirator devices have incorporated a variety of features to address the problems associated with pressure relief. However, it is thematic among such prior art items that they face challenges associated with high pressure and the harm and discomfort that such pressures may cause to the wearer associated with the pressure generated into his or her orifices. More specifically, elevated pressure to the middle ear can lead to hearing loss, hearing difficulty, dizziness, a feeling of fullness in the ear, and/or overall discomfort in the ear. Therefore, it remains desirable to provide an alternative mask concept that mitigates the risk associated with high pressures and the injury that high pressures may cause to the wearer of the mask.

Many masks known in the prior art require a suction action to allow such masks to function as intended. However, the suction action remains undesirable due to the inability of the user to perform suction action on command. More specifically, infants, toddlers, young children, elderly and/disabled persons may lack the ability to perform the suction action on command as associated with prior art devices. Therefore, it remains preferable to have an improved device that does not require a suction action to function.

Medical procedures and devices are known for relieving middle ear pressure due to blockages of the Eustachian tubes, for example the myringotomy surgical procedure, which can lead to scaring of the ear drum with the possibility of permanent hearing loss. An example of a prior art medical device is the Otovent™ medical device, which uses autoinflation of a balloon to create back-pressure to clear the sinuses. The Otovent™, however, requires a thicker balloon material to be able to rush back the air at a high enough pressure as to open the eustachian tube. Because of this, many individuals (including children and adults) have difficulty inflating the balloon to enable use of the device. Additionally, because the device relies on the user to inflate the balloon, the user lacks control over how much constant pressure is even being administered to the eustachian tube. Further, during use by both a wearer and an operator, the device is associated with the challenge of aligning the timing of the administerer deflating the balloon and possibly holding the other nostril of the user shut with the swallowing of the user. Moreover, existing pressure relieving devices such as Otovent™ allow for only the person providing the pressure to perform the necessary action to administer the intended use of the device. This often causes displeasure for the person administering the device and also discomfort for the wearer of the device. Accordingly, there is a need in the art for effective devices that relieve pressure without the disadvantages of the prior art.

Many prior art devices for ear pressure relief additionally exhibit the problem of creating too much pressure. Excess pressure may cause physical harm, such as hearing difficulty, mild hearing loss, dizziness, a feeling of fullness in the ear, or overall discomfort in the ear, to the patient. An improved device to mitigate the risk of creating excess pressure therefore remains desirable.

Some prior art devices for ear pressure relief further exhibit the problem of contamination from a person facilitating the operation of such devices to the wearer of such devices. For example, in prior art devices that are associated with a second person blowing into the device to create pressure, a risk of saliva contamination to the wearer of the mask 101 is accompanied with the operation of the device. In some cases involving devices that have valves or other similar relief mechanisms, the risk of contamination from a third party remains. More specifically, in such devices saliva can transfer to the wearer of the devices from the person applying pressure. Thus, an improved device to mitigate the risk of contamination remains desirable.

Often, prior art devices embody the associated risk of too much pressure because the mechanisms to transfer fluid or gas remain fixedly attached. The rigid attachments therefore prevent an emergency disconnect mechanism in the overpressure scenario. Some other devices exemplify challenges associated with maintaining detachable attachments into the proper configuration or over time to enable the device to properly function. For example, the Nosefrida™ nasal device incorporates injected molded plastic components and molded plastic connections that tend to quickly wear. Such devices especially wear when being contained within a travel case, a likely scenario. Thus, an improved device that does not incorporate fixed attachment mechanisms but still durably functions to accomplish the objectives of the device is desirable.

While in some contexts, pressure relief valves can function to mitigate the risk of overpressure, unique challenges exist in association with the development of an optimized mask 101 to relieve nasal pressure. One such example of a challenge is the risk of mechanical blockages or valve malfunctions caused by the saliva of a second person applying pressure into the mask 101 to allow it to function. Moreover, in many filtration barrier concepts employed in a variety of contexts, such filtration barriers require numerous plastic pieces or components which poses increased risk of additional blockages or inconveniences to the operator or wearer of the mask. Such inconveniences may include, for example, additional failure points which could break during use or storage, more components to relocate and potentially lose during filter replacement, and additional other expense during filter replacement due in part to specialized filter configuration. Thus, an improved device that does not present a high risk of mechanical blockages or valve malfunctioning is desirable.

Many prior art devices feature high durometer rigid tubing. The rigidity of the tubing also presents an added layer of difficulty when mitigating the pressure. High durometer tubing, for example, generally requires more rigidity to the connector and have a much higher “ejection” or relieving pressure. Moreover, typically used high durometer tubing presents additional difficulties associated with packaging. Generally, the inability to coil high durometer tubing into a mask 101 requires additional bulky packaging. Thus, devices that incorporate improvements to the tubing to allow for enhanced pressure relief and packaging remain desirable.

Additionally, many prior art devices fail to allow for a second party to hold the wearer and operate the device. As the wearers of such devices tend to be very young (and often infants), they generally cannot operate the device on their own. Further, they often cry and wiggle during use. This makes the devices very difficult to operate. It remains desirable, therefore, to have an improved device that incorporates features to allow the operator of the device to position the mask 101 while simultaneously holding the wearer of the mask.

Prior art devices often incorporate designs which fail to allow such devices to administer higher pressure quickly. In some cases, such designs fail to have the inner volume within the tube to allow an adult to blow within the tube to administer pressure. Relatedly, small tubes often remain uncomfortable for adult users to blow into to apply pressure. It therefore remains desirable to have an improved device to allow for the comfortable positioning of the tube into a user's mouth prior before and during the pressure delivery.

### SUMMARY OF THE INVENTION

In some embodiments, the invention comprises an overpressure mitigation mask intended to relieve ear pressure in a manner that solves various problems associated with mechanisms known in the prior art. In various embodiments, the mask comprises a connector comprising a barb extrusion mated to an elastomeric low durometer tube. The low durometer tube when exposed to higher pressures is configured to expand during intended use, which prevents harm to the wearer of the mask who otherwise would absorb the pressure. Further in some embodiments, the mask comprises a magnetic connection between the connector piece and the mask which will disconnect when exposed to higher pressures, which without the break point located at the magnetic connector may potentially be harmful to the user.

### BRIEF DESCRIPTION OF THE FIGURES

FIG. 1 depicts a perspective view of the invention.

FIG. 2 depicts the invention in an exemplary use.

### DESCRIPTION OF THE INVENTION

In some embodiments, the invention comprises a mask 101 further comprising a pressure relief valve 102, a magnetic connector 103, a filter barrier 104, low durometer tubing 105, and a mouthpiece 106.

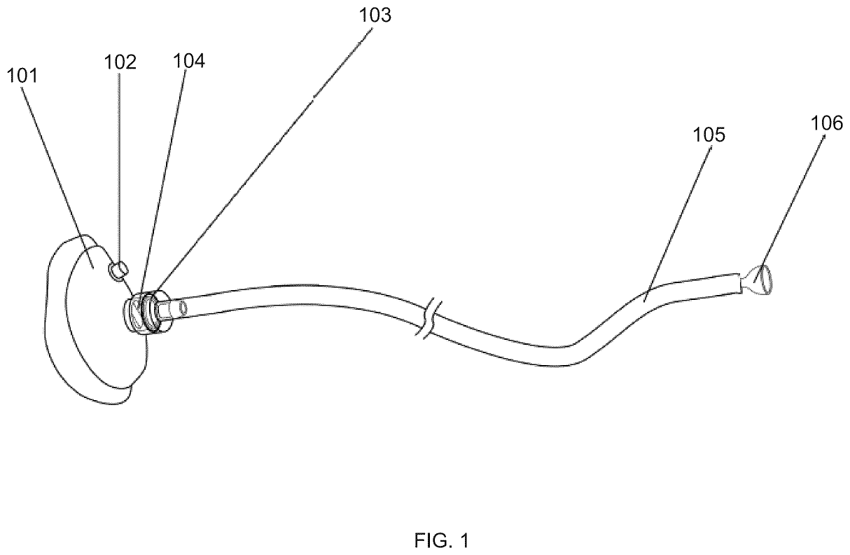

Some embodiments of the invention comprise a mask. In an exemplary embodiment, the mask 101 is configured to cover the nose and mouth with a hermetic seal. In some embodiments, the mask 101 further comprises a pressure relief valve. The pressure relief valve 102 effectively functions to prevent high pressure from causing injury to the wearer of the mask. More specifically, the pressure relief valve 102 provides a safety feature to ensure that the non-wearing user of the mask 101 does not administer too much pressure into the airway of the patient. In order to further address this extreme sensitivity of younger patients, in some embodiments, the pressure relief valve 102 is located as close to the wearer of the mask 101 as possible. In some embodiments, as depicted in FIG. 1, the pressure relief valve 102 is attached to the mask 101 itself. In an exemplary configuration, the pressure relief valve 102 the closest component to the patient (other than aspects of the mask 101 that directly contact the patient). One skilled in the art will recognize that the specific distance will vary minimally between the infant version and the adult versions of the device because the mask 101 size is slightly larger for the adult. In an exemplary embodiment, detection reaction time of the pressure relief valve 102 is improved when the configuration allows for the location of the pressure relief valve 102 close to the patient. This is due to the pressure feedback located at the source (for example, the airway of the wearer of the mask) in order to relieve the pressure and contributes to the effectiveness and immediate responsiveness of the safety-enhancing pressure relief mechanisms. In an exemplary embodiment, risk of harm to the patient can be reduced if the pressure relief valve 102 is closer to the patient. This specifically addresses the risk of harm otherwise present if pressure relief valve 102 were in the tube for example, which could cause limiting or clogging factors in the mask connection, connector piece, or tube itself, which would therefore prevent an accurate reading to the pressure relief valve. This may cause a scenario where the patient may be exposed to higher pressures and the pressure relief valve 102 is not catching it and hence not reliving the pressure needed. The location of the pressure relief valve 102 close to the patient as in the preferred configuration mitigates such risks.

In some embodiments, the mask 101 comprises a magnetic connector. In some embodiments, the magnetic connector comprises a connector piece 103 in the form of a circular mating press fit or magnetic mating attachment to an extruding circular portion of the mask. The connector piece 103 comprises the circular connecting portion on one end and a barb for the tube on the opposite end. The magnetic mate mechanism, configured to separate at high pressure, or in various embodiments at a pressure selected from the range inclusive of 1 psi-5 psi, as will be understood to those skilled in the art, is configured for placement between the mask 101 and the connector piece. In an embodiment, the tube is connected to the barb fitting of the connector piece, this will also disconnect from the mask, but stay with the connector piece. In some embodiments, one magnet comprising the magnetic connector is attached to the outer surface of the mask, and a second magnet is attached to the inner surface of the connecting surface of the low durometer and high diameter tubing or connector piece.

In an exemplary embodiment, the magnets are attached to the extruding interfacing surface of the mask 101 which connects to the connector piece 103 as depicted in FIG. 1. The mating magnet in some embodiments is of dimensions corresponding the inner diameter of the connector piece. The magnets exhibit the desirable characteristic of having a specific magnetic force such that when a pressure exceeding the safe pressure is applied, that the magnetic connector separates the tubing from the mask. In some embodiments, the safe pressure is set at 2 psi, such that the pressure relieving mechanisms to activate at 2 psi. In some embodiments, the mechanism is conservatively configured to activate well below the anticipated rupture pressure of 5.1 psi, however in alternative embodiments the safe pressure is configured at any value in the range inclusive of 1 psi-5 psi. In some embodiments, the magnetic connector provides a safety feature to ensure that the user does not administer too much pressure into the airway of the wearer of the mask. During the intended use, the magnets will disconnect when exposed to a high pressure and relieve the pressure in the device system. The magnets configured as described herein provide a robust connection point to enable the pressure relief mechanisms up to the point at which too much pressure is applied.

The magnetic connector is constructed primarily of a plastic with magnetic inserts. The connector in embodiments intended for adult use has a 30 mm diameter in a circular form, or alternatively a 30 mm by 40 mm substantially oval form, which has the advantageous feature of optimal disconnect at the desired pressure. In alternative uses, including uses intended for children, the connector has a diameter selected from the range inclusive of 5 mm-30 mm.

In an embodiment of the invention, the mask 101 comprises a protrusion. In some embodiments of the invention, the protrusion consists of a barb configured to allow for a strong hold strength between the tube and the connector piece. In the preferred configuration, the barb, in combination with the magnetic mating of the connector piece, exhibits the proper level of strength to hold the tube to the magnetic connector. As such, in such configuration, the barb is not be the weakest joint to disconnect before the magnetic connector. This will allow for the design to reliably break away at the magnets for more accurate pressure relief, and in some embodiments, at a pressure selected from the range inclusive of 1 psi-5 psi. In an alternative embodiment, wherein the magnet mating design is not used, then the barb itself provides a mechanism to control how much pressure will cause the low durometer tube to expand enough to disconnect from the barb. The barb, therefore, in various embodiments acts as either a primary or secondary a pressure relieving feature. The material of the connector piece 103 and barb comprises a injection molded plastic such as polycarbonate, polypropylene, acrylic, nylon, etc. In some embodiments, the diameter of the barb is approximately 20% larger than the inner diameter of the tube.

In some embodiments, the mask 101 comprises a filter barrier. In various embodiments, a purpose of the filter barrier 104 is to block or inhibit exterior substances, for example bodily fluids such as saliva, from being transferred from an operator of the mask 101 to the wearer of the mask. Another purpose of the filter barrier 104 is to prevent external substances from entering the pressure relief valve, which otherwise would risk mechanical blockages to the pressure release valve or other malfunctions. In the preferred configuration, the filter barrier 104 is located between the connection point of the mask 101 and the connector as demonstrated in FIG. 1. The present inventor has recognized the advantage of such placement of the filter barrier 104 in that it allows for minimization of the quantity of needed connection pieces.

In an embodiment filter barrier 104 is configured to cover the entire inner area of the mask. During use of some embodiments, the filter barrier 104 is forced towards the surface of the mask. As such, any saliva or bodily fluids will be stopped by the filter barrier. In various embodiments, the filter barrier 104 is configured such that no room exists around the edges of the filter for fluids to pass through circumventing the filter barrier. In some embodiments, the filter barrier 104 comprises BPA-Free, Latex-Free, Pthalate-Free Polypropylene. In some embodiments, the filter barrier 104 has a thickness of 0.5″, though in various embodiments the thickness of the filter barrier 104 is chosen from the range inclusive of 0.2″-0.8″.

In some embodiments, the mask 101 comprises low durometer tubing 105. Low durometer tubing 105 exhibits the advantageous and over-pressure mitigating characteristic of expansion, providing pressure relief and therefore preventing harm to the wearer of the mask. As pressure increases above the safe amount, the tubing becomes ejected or disconnected from the barb of the connector piece 103 and hence, the low durometer tube in combination with the barb acts as a pressure relieving feature. In some embodiments, the larger diameter of the low durometer tubing 105 further exhibits the advantageous characteristic of allowing for increased airflow, enhancing the ease of use for the non-wearer operator of the mask during the application of pressure, optionally by blowing into the tubing). The durometer of the tube is specifically configured in some embodiments to prevent too much rigidity to the magnetic connector and enable the optimal “ejection” or disconnection of the tube to the magnetic connector.

The low durometer tubing 105 connects to the magnetic connector in some embodiments by a protrusion, optionally a barb. In the preferred configuration, low durometer tubing 105 comprises the materials of medical-grade BPA free silicone. In the preferred configuration, the durometer of the low durometer tubing 105 is 50 Shore A. In varying embodiments, the durometer of the low durometer tubing 105 is selected from the range inclusive of 20 Shore A to 80 Shore A. In varying embodiments, the durometer is selected from the range that covers soft to medium hard silicone. In the preferred configuration, the diameter of the low durometer tubing 105 is 0.375″. In varying embodiments, the diameter is selected from the range inclusive of 0.25″-0.5″. In some cases, using a higher diameter allows for higher pressure to be administered quickly by the non-wearing operator of the mask. During the intended use, the low durometer tubing 105 transfers pressure applied to the system to the wearer of the mask, while enabling the appropriate risk mitigation via the pressure relief mechanisms if too much pressure is applied. Additionally, low durometer tubing 105 has other advantages in that it allows for easier connectivity to the barb of the connector and provides for increased flexibility leading to easier storage.

More specifically, the low durometer of the tubing allows for it to retain enhanced flexibility. In some embodiments, the low durometer tubing 105 coils easily into the mask. Thus, a much slimmer, more compact and more secure packaging configuration is available for use with the preferred configuration.

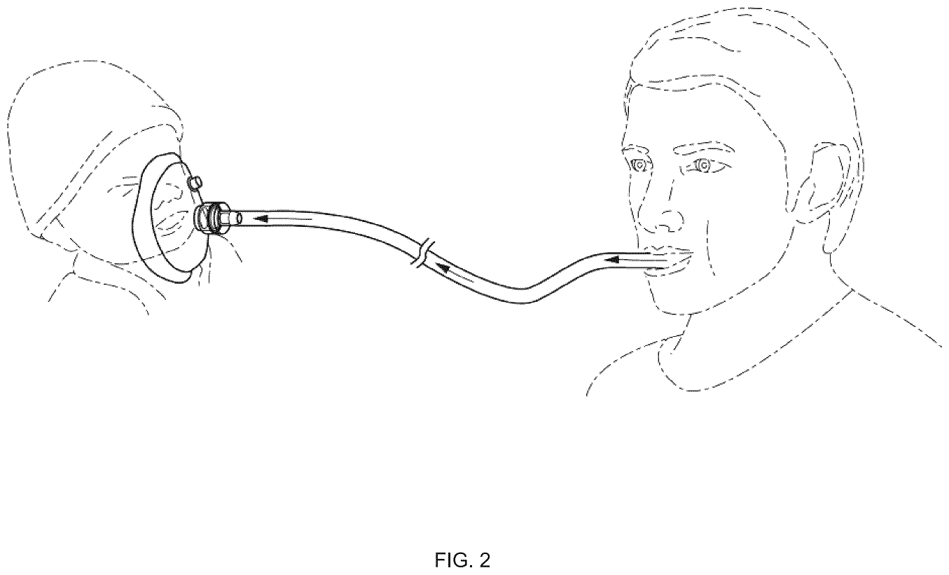

Additionally, the low durometer tubing 105 allows for more flexibility when using the device, particularly with very young children. In an example of use where the intended wearer of the mask 101 is a baby, who (as is a typical case) is crying uncontrollably and not wanting to stay still, the non-wearing operator of the mask may harness the child with one hand while positioning the mask with the other, due specifically to the intended flexibility of the mask in the preferred configuration. As shown in FIG. 2, the low durometer tubing 105 is placeable into the mouth of the non-wearing operator of the mask 101, optionally by use of a mouthpiece 106, and ready for the application of pressure. Such configuration solves a commonly faced challenge of other prior art attempts, which due to the lack of flexibility in their designs prevent the non-wearing operator of the mask 101 from effectively deploying to an infant while holding the infant.

In some embodiments, the mask 101 comprises a mouthpiece 106 for the non-wearing operator of the mask 101 which comprises a high-diameter low durometer tubing, which in some embodiments of the invention is up to 0.5 inches inner diameter. This addresses the problem of the tubing 105 having too small of a diameter which becomes an acute challenge when the non-wearing operator of the mask 101 engages in forceful blowing through the tube to administer pressure to the wearer of the mask. Therefore, more ergonomic mouthpiece 106 is needed to comfortably position the tube in the non-wearing operator's mouth before and during pressure delivery. In the preferred configuration, the mouthpiece 106 is configured to press fit into the low durometer tubing 105. The mouthpiece 106 is further configured for placement into the inside of the operator's mouth. In some embodiments, the mouthpiece 106 will be approximately 0.5″ of width, which is sufficient to allow the user to properly grip the mouthpiece 106 to prevent unintentional ejection from the mouth during application of positive pressure air. In an exemplary configuration, the mouthpiece 106 further comprises a rigid section, optionally featuring ridges and/or texture, to allow the operator to grip the mouthpiece 106 with his or her teeth during operation.

In some embodiments, there is a stronger connection point at the connection point of the mouthpiece 106 to the low durometer tubing 105 than to any other connection point associated with the mask 101. This need stems from the risk posed by alternative potential configurations that a weak connection at the mouthpiece-low durometer tubing 105 interaction point, more specifically the risk that the mouthpiece 106 and tube may fall out during use. For example, if the wearer of the mask 101 (often an inconsolable infant) must be restrained during use, the mouthpiece 106 and low durometer tubing 105 may more easily be dislodged. A stronger connection point at the interaction of the mouthpiece 106 and the low durometer tubing 105 prevents the dislodging during intended usage while preserving the desirable over-pressure mitigation characteristics of the mask, particularly those associated with the magnetic connection, the pressure relief valve, and the low durometer tubing.

In the foregoing specification, specific embodiments have been described. However, one of ordinary skill in the art appreciates that various modifications and changes can be made without departing from the scope of the disclosure as set forth in the claims to follow in a subsequent disclosure. Accordingly, the specification and figures are to be regarded in an illustrative rather than a restrictive sense, and all such modifications are intended to be included within the scope of present teachings.

The benefits, advantages, solutions to problems, and any element(s) that may cause any benefit, advantage, or solution to occur or become more pronounced are not to be construed as a critical, required, or essential features or elements of any or all subsequent claims.

Any noun in the singular is also intended to encompass the noun in the plural and vice versa, unless specifically stated as otherwise intended. Any pronoun or other identifier in the female form is also intended to encompass the pronoun or other identifier in the male form and vice versa, unless specifically stated as otherwise intended. The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially”, “essentially”, “approximately”, “about” or any other version thereof, are defined as being close to as understood by one of ordinary skill in the art, and in one non-limiting embodiment the term is defined to be within 10%, in another embodiment within 5%, in another embodiment within 1% and in another embodiment within 0.5%. The terms “coupled,” “connected” and “linked” as used herein is defined as connected, although not necessarily directly and not necessarily mechanically. A device or structure that is “configured” in a certain way is configured in at least that way, but may also be configured in ways that are not listed.

While the foregoing is directed to aspects of the present disclosure, other and further aspects of the disclosure may be devised without departing from the basic scope thereof.

What is claimed:

1. An ear pressure relieving device, comprising:
a mask to be worn over the nose and mouth of a target wearer, the mask including a connector and a filter barrier; and
wherein low durometer tubing is attached at a first end to the connector of the mask and a mouthpiece is attached to a second end of the low durometer tubing.

2. The ear pressure relieving device of claim 1, wherein the mask includes a pressure relief valve.

3. The ear pressure relieving device of claim 1, wherein the connector is a magnetic connector.

4. The ear pressure relieving device of claim 3, wherein the magnetic connector comprises a barb at one end and a circular magnet at the other end, wherein the first end of the low durometer tubing is attached to the barb.

5. The ear pressure relieving device of claim 1, wherein the connector releases at a breakaway pressure between 1 and 5 psi.

6. The ear pressure relieving device of claim 1, wherein the filter barrier comprises BPA-Free, Latex-Free, Pthalate-Free Polypropylene.

7. The ear pressure relieving device of claim 1, wherein the low durometer tubing comprises a durometer between 20 Shore A to 80 Shore A.
